(19) United States
(12) Patent Application Publication
(10) Pub. No.: US 2008/0194883 A1
(43) Pub. Date: Aug. 14, 2008

(54) Menthol-Containing Solids Composition

(75) Inventors: Reinhard Nowak, Mt. Pleasant, SC (US); Michael Michler, Eimen (DE); Ingo Reiss, Holzminden (DE); Robert Winkel, Wuppertal (DE)

Correspondence Address: ROYLANCE, ABRAMS, BERDO & GOODMAN, L.L.P., 1300 19TH STREET, N.W., SUITE 600, WASHINGTON,, DC, 20036, US

(73) Assignee: SYMRISE GMBH & CO. KG, Holzminden (DE)

(21) Appl. No.: 11/910,809

(22) PCT Filed: Apr. 6, 2006
(86) PCT No.: PCT/EP06/61392

§ 371 Date: Apr. 15, 2008

Related U.S. Application Data
(60) Provisional application No. 60/668,782, filed on Apr. 6, 2005.

(51) Int. Cl. C07C 35/12

(52) U.S. Cl. 568/829

(57) ABSTRACT

A menthol-containing solids composition comprising or consisting of:
(a) a solid menthol component consisting of bodies having a content of 95 wt. % or more of menthol, based on the total weight of the bodies, and
(b) a solid anticaking agent, comprising or consisting of particles having a particle size of not more than 100 μm,
wherein particles of the anticaking agent adhere to the surface of the bodies of the menthol component,
wherein the solid anticaking agent is chosen from the group consisting of
calcium phosphate, magnesium phosphate,
magnesium hydroxycarbonate,
magnesium oxide,
mixtures of magnesium oxide and silicon dioxide,
mannitol,
calcium silicate, magnesium silicate, talc,
polydimethylsiloxane,
Al, Ca, K, Na, Mg or NH₄⁺ salts of edible fatty acids,
and mixtures thereof,
and wherein the content of the anticaking agent is not more than 8 wt. % and the content of the menthol component is at least 91 wt. %, in each case based on the total weight of the solids composition.

The invention relates to a menthol-containing solids composition, articles comprising such a solids composition, the use of such a solids composition and a process for the preparation thereof.

The menthol-containing solids compositions is preferably free-flowing.

I-Menthol has a unique refreshing taste, a minty smell and a strong cooling effect on skin and mucous membrane. It is used, for example, in oral care, in cosmetic and pharmaceutical preparations, in tobacco and in confectionery, as described e.g. in Perfumer&Flavorist, vol. 13, October-November 1988, p. 37.

I-Menthol is the main constituent of peppermint oils from Mentha arvensis (content: 70 to 80%) and Mentha piperita (content: 50 to 60%). I-Menthol is obtained from crude peppermint oil by crystallization. The crystals vary in taste and crystal size and shape, depending on the crystallization process and starting material (Perfumer&Flavorist; vol. 22, November-December 1997, p. 1). A small residual amount of liquid peppermint oil adheres to these menthol crystals obtained from peppermint oils (the content of I-menthol is conventionally not more than 99.2 wt. %), inhibiting caking or formation of lumps of the menthol crystals, but also noticeably influencing the sensory profile.

Many processes are known for the preparation of synthetic menthol. An economical preparation of synthetic I-menthol, for example, starts from thymol. From the eight stereoisomeric menthols formed by hydrogenation, I-menthol is obtained in a chemical purity of >99% and an enantiomer purity of >99% via several process steps (e.g. in Bauer, Garbe, Surburg, Common Fragrance and Flavor Materials, 4^{th} ed., Wiley-VCH, Weinheim 2001, p. 52-55). The I-menthol resulting from this process is distinguished sensorially by its purity and its radiant power.

Menthol, in particular I-menthol, is commercially obtainable in various solid forms, and examples of conventional forms are powders, crystals, solidified distillate, flakes and pressed pieces.

Synthetic I-menthol prepared and crystallized according to the processes of DE-A 2109456, DE-A 2530481 and EP 0 909 205 (having a melting point of 42-43° C.) is commercially available in the form of white crystals or in the form of pressed pieces (pellets) as well as a solidified distillate (Symrise GmbH & Co. K G, Holzminden).

Solid forms of menthol known to date, in particular of menthol which is free from peppermint oil, form lumps during storage after a longer period of time, as a result of which they lose their pourability and ease of handling.

U.S. Pat. No. 3,064,311 describes I-menthol in flaked form. For its preparation, distilled I-menthol is melted and a thin molten film layer is applied to a supercooled surface. The solidified I-menthol film is broken up into small parts. The product of this process is a fragile I-menthol flake which has a thickness of from 0.125 to 1.25 mm and a spread of from 3 to 25 mm. The flaked I-menthol prepared in this way shows, as described in the US specification, adhesion and formation of lumps of the flakes after 24 hours.

WO 03/101924 discloses menthol pressed pieces having a content of alpha-menthol of greater than or equal to 70 wt. %, which show a comparatively quite low tendency towards caking and formation of lumps. However, during longer storage times of more than three months, these pressed pieces also gradually form lumps and cake.

JP 08-020549 relates to powders containing I-menthol which comprise about 90 wt. % of synthetic I-menthol (particle size 50-200 μm) and about 10 wt. % of silica gel (particle size of less than 10 μm, preferably in the range of 2-5 μm). This product (bulk height: 50 cm) showed no caking after storage for one month. For the preparation of the product, a surfactant (e.g. 2 wt. % of decaglyceryl monolaurate, based on the total amount of menthol and silica gel) was first introduced into water at a temperature above the melting point of I-menthol (42° C.). Silica gel and molten menthol or menthol on silica gel were then introduced successively into the mixture of surfactant and water at 50° C. After brief stirring, this mixture was cooled with ice-water, washed with water, filtered off and dried.

However, our own experiments have shown that the product prepared according to JP 08-020549 has a water content of up to 3 wt. % after the drying. As already mentioned in the introduction of JP 08-020549, moisture leads to a lowering of the melting point of the menthol and to a certain tendency towards caking; the water content is therefore a disadvantage. The residual content of surfactant in the pulverulent menthol-containing product described is furthermore undesirable. In addition, the high content of silica gel in the menthol-silica gel powder disclosed is a disadvantage; inter alia the silica gel imparts an unpleasant sensory impression in the mouth.

The object of the present invention was to provide a solids composition having a high content of menthol, wherein the composition should have an improved, very high storage stability, be simple to prepare and easy to handle, and preferably have a good (“free-flowing”) pourability, resulting in an easy processability.

This object was achieved according to the invention by a menthol-containing solids composition comprising or (preferably) consisting of

(a) a solid menthol component consisting of bodies having a content of 95 wt. % or more of menthol, based on the total weight of the bodies, and

(b) a solid anticaking agent, comprising or (preferably) consisting of particles having a particle size of not more than 100 μm,

wherein particles of the anticaking agent adhere to the surface of the bodies of the menthol component,

wherein the solid anticaking agent is chosen from the group consisting of

calcium phosphate, magnesium phosphate,

magnesium hydroxycarbonate,

magnesium oxide,

mixtures of magnesium oxide and silicon dioxide,

mannitol,

calcium silicate, magnesium silicate, talc,

polydimethylsiloxane,

Al, Ca, K, Na, Mg or NH₄⁺ salts of edible fatty acids

and mixtures thereof,

and wherein the content of the anticaking agent is not more than 8 wt. % and the content of the menthol component is at least 91 wt. % (preferably at least 92 wt. %), in each case based on the total weight of the solids composition.

In this context, the menthol-containing solids composition according to the invention is preferably a free-flowing composition.

In the present text, all references to the presence of a material in the solid state of aggregation (“solids composition”, “solid component”, solid form” or the like) relates to the state at 20° C. under 1,013 mbar. The same applies to references to “free-flowing” compositions.

The invention is based on the surprising finding that it is possible to provide a menthol-containing solids composition having a high content of menthol which has an extremely high storage stability, that is to say a high resistance towards formation of lumps and caking, by mixing an appropriate menthol component with a solid anticaking agent from the abovementioned group. In this context, the solid anticaking agent must comprise particles having a particle size of not more than 100 μm or consist of such particles. Particles of the anticaking agent adhere to the surface of the bodies of the menthol component,

The solid anticaking agents to be employed according to the invention are indeed already known as anti-caking agents, but this alone does not yet qualify them for the use according to the invention. In our own studies, it has in fact been found that not just any desired conventional anti-caking agent can be employed in order to achieve the object on which the invention is based. For example, menthol-containing solids compositions which comprised, instead of the anticaking agents to be employed according to the invention, in each case 8 wt. %, based on the total weight of the solids composition, of other anti-caking agents (such as e.g. aluminium silicate, bentonite, calcium carbonate, sodium carbonate, sodium silicate, cellulose) proved to be not stable to storage.

The precise reason for the success according to the invention in the use of the solid anticaking agents described above is not known in detail. However, there is presumably a particular interaction, which was not to be expected beforehand, between menthol and the anticaking agent to be employed according to the invention, which renders it possible for the anticaking agent present according to the invention to adhere to the surface of the bodies of the menthol component to an extent such that the sublimation capacity or the evaporation of the menthol is reduced in the long term. Our own studies have shown that the caking and formation of lumps of conventional menthol forms is greatly promoted by sublimation or by evaporation and redeposition of the menthol. Particles of the anticaking agents to be employed according to the invention having a particle size of not more than 100 μm, preferably of not more than 50 μm, and particularly preferably of not more than 20 μm, are apparently outstandingly capable of adhering to the surface of bodies of the menthol component and of limiting in the long term the “free” menthol surface available for sublimation or evaporation.

Preferably, the solid anticaking agent to be employed according to the invention is chosen from the group consisting of

calcium phosphate, magnesium phosphate,

magnesium hydroxycarbonate,

magnesium oxide,

mixtures of magnesium oxide and silicon dioxide,

calcium silicate, magnesium silicate, talc,

polydimethylsiloxane,

Ca, K or Na salts of edible fatty acids

and mixtures thereof.

Particularly preferred solid anticaking agents are chosen from the group consisting of

calcium phosphate, magnesium phosphate,

magnesium hydroxycarbonate,

magnesium oxide,

mixtures of magnesium oxide and silicon dioxide,

calcium silicate, magnesium silicate

and mixtures thereof.

The menthol-containing solids compositions according to the invention are exceptionally stable to storage and are still flowable even after long periods of time; when the particularly preferred solid anticaking agents (see above) were employed, no caking or formation of lumps was to be found in our own studies after a storage time of more than 6 months at 20° C. and a bed height of 1 m. In this context, a solid menthol component consisting of bodies in the form of crystals or pressed pieces was employed. The menthol-containing solids composition according to the invention which was investigated remained convenient and reliable to handle and pourable, flowable and meterable over the entire observation period.

Table 1 shows the results of a flowability investigation after storage for three months at 20° C. with menthol crystals (bulk height: 50 cm) having a particle size distribution according to Example 1a below. The percentage contents of the particular anticaking agent relate to the menthol-containing composition consisting of menthol crystals and anticaking agent.

TABLE 1

| | 0.5 wt. % of | 1 wt. % of | 2 wt. % of | 4 wt. % of |
|---|---|---|---|---|
| | anticaking | anticaking | anticaking | anticaking |
| | agent | agent | agent | agent |
| Magnesium silicate | C | B | B | A |
| Magnesium | C | B | A | A |
| hydroxycarbonate | | | | |
| pentahydrate | | | | |
| Calcium silicate | B | B | A | A |
| (200 μm) | | | | |
| Magnesium oxide, | A | A | A | A |
| nanopowder | | | | |
| (~30 nm, >50 m²/g) | | | | |
| Magnesium oxide, | A | A | A | A |
| powder and silicon | | | | |
| dioxide (200 m²/g) | | | | |
| (weight contents 2:1) | | | | |

The data in parentheses relate to the specific surface area in m²/g measured by the BET method, or the average particle size in nm (nanometers) or μm (micrometers).
D = severe lump formation, no flowability
C = moderate lump formation, poor flowability
B = free flowability, scarcely any lump formation
A = very good flowability, no lump formation

Magnesium oxide is a very particularly preferred solid anticaking agent, since the best results have been achieved with this. Very good results are achieved in particular with very finely divided magnesium oxide having average particle sizes of less than or equal to 300 nm (nanopowder); the average particle size is preferably less than or equal to 100 nm, and the average particle size is particularly preferably in the range of from 5 to 50 nm. The use of magnesium oxide having a specific surface area in the range of from 10 to 200 m²/g is preferred, preferably in the range of from 30 to 150 m²/g (BET surface area, measured in accordance with DIN 66131). Various qualities of such magnesium oxide powders are commercially available.

Menthol-containing compositions with magnesium oxide or mixtures of magnesium oxide and silicon dioxide as the solid anticaking agent showed no formation of lumps or caking even after a storage time of more than nine months.

If mixtures of magnesium oxide and silicon dioxide are employed as the solid anticaking agent, the weight ratio of magnesium oxide to silicon dioxide is preferably in the range of from 10:1 to 1:10, preferably in the range of from 5:1 to 1:5, and particularly preferably in the range of from 3:1 to 1:3.

Preferably, the menthol-containing solids composition according to the invention is formed by a procedure in which particles of the solid anticaking agent adhere to the surface of the bodies of the menthol component and cover this in a manner such that the sublimation capacity or the evaporation of the menthol from the bodies of the menthol component at 25° C. under 1,013 mbar is reduced by at least 25%, preferably at least 40%, compared with the menthol component (a). This reduction in the sublimation rate or the evaporation rate can be determined, for example, by means of thermogravimetry in accordance with DIN 51006. In thermogravimetry, in the present case the weight of the non-mixed menthol component (a) and of a menthol-containing solids composition according to the invention is measured as a function of time at the same constant temperature (i.e. isothermally) below the melting point, preferably at 25° C. (and under 1,013 mbar).

Our own microscope photographs have shown that in preferred menthol-containing solids compositions according to the invention, at least 80%, preferably at least 90%, and particularly preferably at least 95% of the surface of the solid menthol component (a) is covered with the solid anticaking agent.

A process according to the invention for the preparation of a menthol-containing solids composition according to the invention is very easy to carry out. It comprises the following steps:

mixing of

(a) a solid menthol component consisting of bodies having a content of 95 wt. % or more of menthol, based on the total weight of the bodies, and

(b) a solid anticaking agent, comprising or consisting of particles having a particle size of not more than 100 μm,

until particles of the solid anticaking agent adhere to the surface of the bodies of the menthol component,

wherein the solid anticaking agent is chosen from the group consisting of

calcium phosphate, magnesium phosphate,

magnesium hydroxycarbonate,

magnesium oxide,

mixtures of magnesium oxide and silicon dioxide,

mannitol,

calcium silicate, magnesium silicate, talc,

polydimethylsiloxane,

Al, Ca, K, Na, Mg or NH₄⁺ salts of edible fatty acids

and mixtures thereof,

and the content of the solid anticaking agent is not more than 4 wt. % and the content of the menthol component is at least 95 wt. %, in each case based on the total weight of the solids composition prepared.

The above statements apply again in respect of the solid anticaking agents which are preferably to be employed.

The solid menthol component according to the invention which is employed consists of bodies having a content of 95 wt. % or more of menthol, based on the total weight of the bodies. Preferably, however, the menthol content is greater than or equal to 98 wt. %, a menthol content of greater than or equal to 99 wt. % is very particularly preferred, and a menthol content of greater than or equal to 99.5 wt. % is especially preferred, in each case based on the total weight of the bodies of the solid menthol component.

From that stated above, it can be seen that the bodies of the menthol component conventionally comprise no inorganic constituents, that is to say, in particular, no water and also no anticaking agent.

In the individual case, the presence of small amounts of inorganic constituents in the menthol component bodies present according to the invention, however, may be acceptable or even desired. Preferably, the content of inorganic constituents is then in the range of between 0 and 1%, based on the total weight of the bodies. In a particularly preferred embodiment, the menthol-containing solids composition according to the invention comprises not more than 0.2 wt. % of water, based on the total weight of the solids composition.

The form of the bodies of the menthol component (a) in a menthol-containing solids composition according to the invention can be varied within wide ranges. In particular, the bodies of the menthol component can be in the form of crystals, prills, flakes, pressed pieces, pastilles, granules, powders and/or dust. Those forms which tend particularly severely towards caking in the absence of the solid anticaking agent (b) present according to the invention are particularly to be emphasized, that is to say menthol bodies in the form of crystals, prills or flakes.

Particularly preferred menthol-containing solids compositions according to the invention comprise (i) no peppermint oil on the surface of the bodies of the menthol component (in contrast to conventional menthol products which are isolated from naturally occurring mixtures) and/or (ii) no surfactants, that is to say no surface-active substances, and/or (iii) not more than 0.2 wt. % of water, based on the total weight of the solids composition (in contrast to the products according to JP 08-020549).

In the menthol-containing solids composition according to the invention, the menthol employed can be d-menthol, I-menthol and any desired mixture thereof; I-menthol, d-menthol and racemic menthol are preferred and I-menthol is particularly preferred.

The menthol employed can be of synthetic or natural origin, but the use of synthetic menthol is preferred. In particular, if menthol of natural origin is employed, it is difficult to ensure the high purity often desired for the menthol (see the above statements).

Any desired mixtures of synthetic and natural menthol and of racemic and enantiomerically pure menthol can also be employed.

If menthol crystals, preferably needle-shaped crystals, are employed as the menthol component, these preferably have crystal sizes of up to 2 mm, and crystal sizes in the range of from 50 to 1,500 μm are preferred. Particularly preferably, more than 75 wt. % of the crystals have a crystal size in the range of from 50 to 1,000 μm (1 mm), and in this context in turn preferably more than 60 wt. % of the crystals have a crystal size in the range of from 200 to 800 μm (0.2 to 0.8 mm), the contents in each case being based on the total weight of the menthol crystals.

A typical crystal size distribution (according to sieve analysis) of menthol crystals which are particularly preferably employed for the preparation of a menthol-containing solids composition according to the invention is the following:

2-25 wt. % having a crystal size of less than 0.20 mm;

40-80 wt. % having a crystal size in the range of from 0.20 mm to 0.50 mm;

5-35 wt. % having a crystal size in the range of from 0.51 mm to 0.80 mm;

1-15 wt. % having a crystal size in the range of from 0.81 mm to 1.0 mm;

0.2-8 wt. % having a crystal size in the range of from 1.1 mm to 1.25 mm; and

0.2-10 wt. % having a crystal size in the range of from 1.26 mm to 2 mm.

In the preparation of a menthol-containing solids composition according to the invention, a change in the crystal size and crystal size distribution may occur, chiefly if menthol crystals, in particular needle-shaped menthol crystals, are employed. Depending on the nature and type of the mixer used and the mixing intensity and duration of mixing, a menthol-containing solids composition in which the menthol crystals overall are smaller than those of that originally employed before the mixing operation is obtained.

As already mentioned, bodies of the menthol component can be in many forms, for example, in addition to the crystalline form, in the form of prills, flakes, pressed pieces, pastilles, granules, powders and/or dust.

Prills are small beads having a diameter in the range of from 0.2 to 1.5 mm, preferably in the range of from 0.3 to 1 mm. Prills are obtained, for example, by forming drops of liquid menthol and then cooling the menthol drops in a cooling medium (e.g. cold air).

Flakes are obtained, for example, by solidification of a menthol melt on a cooling roll. These flakes conventionally have a flake thickness of from 0.5 to 2 mm, and in this context the flake area is variable in shape. The flake area is regularly in the range of length: 5 to 15 mm, width: 2 to 10 mm.

Pressed pieces can be in the form of, for example, beads, cubes, parallelepipeds, pillows, cylinders, tablets, pellets or briquettes. In this context, the pressing (compacting) of the menthol can be carried out in the most diverse manner and way. Proven pressing processes are described, for example, in Chemie-Anlagen+Verfahren 1985, volume 4, pages 51, 54 and 59. The dimensions of the pressed pieces can differ widely. In the case of a bead, the diameter is in the range of from 1 mm to 40 mm; preferably in the range of 5 to 20 mm. In the case of a pellet, the length and width are in the range of from 3 to 30 mm, preferably in the range of from 5 to 15 mm. The height is in the range of from 0.5 to 20 mm, preferably in the range of 2 to 8 mm.

During the compacting, a pressing force in the range of from 5 to 200 kN (kilonewton) is conventionally applied in the compactor apparatus. A pressing force of from 10 to 100 kN is preferred, particularly preferably from 20 to 80 kN. During the compacting of the menthol, linear pressing forces of 1-5 newton/mm·mm, based on the diameter, are preferred.

In the context of the present text, pastilles are understood as meaning the menthol forms obtained by dropping a menthol melt on to a cooled, non-curved cooling surface (e.g. cooling belt, cooling plate).

Granules are, for example, menthol forms which are obtainable by breaking or other types of fragmentation, which can as a result be entirely different in shape and size (e.g. broken or crushed granules).

Powders and dust of the menthol are obtained e.g. either as a by-product during the crystallization of natural I-menthol, or can be a constituent in products obtained by grinding larger menthol bodies.

According to the invention, the content of the solid anticaking agent is not more than 8 wt. %, based on the total weight of the solids composition. Preferably, the content of the solid anticaking agent is not more than 4 wt. %, preferably not more than 2.5 wt. %, and particularly preferably in the range of from 0.2 to 2 wt. %, based on the total weight of the solids composition.

In a menthol-containing solids composition according to the invention, the content of the menthol component is at least 95 wt. %, based on the total weight of the solids composition. Particularly preferably, however, the content of the menthol component is in the range of from 96 to 99.8 wt. %, very particularly preferably in the range of from 98 to 99.8 wt. %.

It has already been stated above that the solid anticaking agents to be employed according to the invention are far more suitable than other substances for covering the surface of menthol bodies such that the sublimation capacity or the vapour pressure of the menthol bodies is reduced. It therefore goes without saying that the preferred content of the solid anticaking agent in a menthol-containing solids composition according to the invention depends on the form in which the bodies of the menthol component are present, since different values result for the ratio of the surface of the bodies to the weight of the bodies, depending on the shape of the bodies.

A menthol-containing solids composition according to the invention having a menthol component which consists of menthol crystals preferably comprises 0.5 to 2.5 wt. %, preferably 0.5 to 2.0 wt. % of the solid anticaking agent.

A menthol-containing solids composition according to the invention, the menthol component (a) of which consists of menthol pressed pieces or menthol flakes, preferably comprises 0.2 to 1 wt. % of the solid anticaking agent.

A menthol-containing solids composition according to the invention having a menthol component which consists of menthol prills preferably comprises 0.3 to 1.5 wt. % of the solid anticaking agent.

In a mixture of magnesium oxide and silicon dioxide as the solid anticaking agent (b) which is preferably to be employed according to the invention, the particles of the silicon dioxide preferably consist of precipitated silica and/or pyrogenic silicon dioxide. The use of pyrogenic silicon dioxide is preferred somewhat to that of precipitated silica.

The use of (preferably pyrogenic) silicon dioxide having a specific surface area in the range of from 50 to 500 m²/g is preferred, preferably in the range of from 100 to 400 m²/g (BET surface area, measured in accordance with DIN 66131).

Precipitated silicas (empirical formula: SiO₂) can be prepared, for example, by means of precipitation processes or hydrothermal processes and are available in various qualities, inter alia, from Degussa-Hüls A G under the trade names Sipernat® or Carplex®. Precipitated silicas advantageously have a specific surface area in the range of from 50 to 500 m²/g, measured by the BET method, and particle sizes in the range of from 3 to 100 μm. Preferred precipitated silicas are amorphous and have particle sizes in the range of from 3 to 50 μm.

Amorphous, highly disperse pyrogenic silicon dioxide can be prepared, for example, by means of an oxyhydrogen gas flame or in an electric arc. Highly disperse hydrophobic or hydrophilic silicon dioxides are obtainable in various qualities, for example, from Degussa-Hüls A G under the trade name Aerosil®. The size of the spherical primary particles here is regularly in the range of from 7 to 40 nm. These primary particles form agglomerates and aggregates (see DIN 53206) and have no defined agglomerate size, in contrast to precipitated silicas. Preferably, the highly disperse silicon dioxide has a specific surface area in the range of from 50 to 400 m²/g, preferably in the range of from 100 to 300 m²/g, measured by the BET method. The average size of the primary particles is preferably in the range of from 7 to 20 nm, preferably in the range of from 7 to 16 nm.

After 2 hours at 105° C. (in accordance with DIN ISO 787/II), the pyrogenic silicon dioxides show a loss on drying of not more than 2 wt. %. After 2 hours of ignition at 1,000° C., the pyrogenic silicon dioxides have a content of SiO₂ of at least 99.8 wt. %. The sieve residue (according to Mocker, 45 μm; in accordance with DIN ISO 787/XVIII) is less than 0.1 wt. %.

The silanol groups on the particle surface can be free (hydrophilic silicon dioxide) or can be derivatized with silanes or silazanes (hydrophobic silicon dioxide).

Further information on the silicon dioxides which can be employed in the context of the invention can be found, for example, in EP 0 331 425 and the Sivento product brochure on “Aerosil®-Pyrogene Kieselsäuren [Aerosil®-Pyrogenic Silicas]” from Degussa-Hüls A G.

The menthol-containing solids compositions according to the invention have an outstanding flowability even after a long storage time.

The flowability of bulk goods can be characterized by the angle of repose. At an angle of repose of less than 30°, “very readily flowing” is referred to, at an angle of repose in the range of from 30° to 45°, “free-flowing” is referred to, and at an angle of repose of greater than 45°, “poorly flowing” is referred to. In this context, it is to be noted that no angle of repose at all can be determined on forms with severe formation of lumps or caking, since in such cases there is no flowability. The angle of repose can be determined in accordance with DIN ISO 4324.

Menthol-containing solids compositions according to the invention in which the solid anticaking agent and the menthol component are chosen and are matched to one another in their amounts such that the solids composition is “very readily flowing” or at least “free-flowing” are preferred.

Surprisingly, the menthol-containing solids compositions according to the invention contribute towards solving a further problem which is of high importance in practice. In fact, it was hitherto a disadvantage of products comprising menthol in high concentrations that the menthol crystallizes out of or in these products. Ready-to-use liquid flavor compositions having a high menthol content, in particular for use in the field of oral care, are prepared, for example, by mixing up to 50 wt. % of solid menthol with liquid peppermint oils. In this procedure, the solid menthol is incorporated into the flavor composition for intensification of the fresh, mentholic taste impression, and is thereby dissolved (e.g. in the liquid peppermint oil). It was hitherto a problem that during storage at relatively low temperatures and/or during storage over a relatively long period of time, menthol crystallizes out of such flavor compositions having a high menthol content, so that a heterogeneous product forms. As a result, inter alia, the sensory impression of such products changes adversely. Surprisingly, in products into which a menthol-containing solids composition according to the invention has been incorporated, there is only a very low or even no tendency towards crystallization of menthol; an increased storage stability in respect of the menthol constituent of such products can thus be achieved when the menthol-containing solids composition according to the invention is employed. Preferably, in this context the bodies of the menthol component of the menthol-containing solids composition according to the invention are in the form of crystals.

Menthol-containing solids compositions according to the invention, in particular those in which the bodies of the menthol component are in the form of crystals, dissolve rapidly in water, ethanol and mixtures thereof, also without an additional supply of heat (in this context, see Example 2). The solids compositions according to the invention can thus be incorporated directly into a product, without having to be diluted or dissolved beforehand. The rapid solubility is of advantage, inter alia, if a rapid distribution and/or dissolving of the menthol is desired or necessary, such as, for example, in the preparation of flavors, chewing gums, sweets or toothpastes. Thus, in the preparation of products, it is possible to incorporate the menthol-containing solids composition according to the invention into a product separately from the remaining flavor and as a result to reduce the times for mixing or kneading. For example, in the preparation of a ready-to-use toothpaste which comprises 1 wt. % of a toothpaste flavor which in turn comprises 40 wt. % of solid menthol, the dosage of the toothpaste flavor without solid menthol can be reduced to 0.6 wt. % and a content of 0.4 wt. % can be added directly to the toothpaste mixture in the form of a menthol-containing solids composition according to the invention.

A menthol-containing solids composition according to the invention moreover shows unexpected sensory effects.

In chewing gum strips/dragées, the incorporation of a menthol-containing solids composition according to the invention had the effect of a delayed release of the flavor and therefore an increased “long-lasting” effect during chewing, that is to say the flavor is perceived for a longer period of time, compared with incorporation of the usual I-menthol crystals at the same dosage (1 wt. %, based on the finished chewing gum). Furthermore the flavor profile of chewing gums into which a content of menthol-containing solids compositions according to the invention was incorporated displayed a clearer and more radiant menthol taste by comparison.

A combination of the incorporation of (i) spray-dried menthol (e.g. 0.7 wt. %, based on a finished chewing gum), which ensures a balanced fresh and rapid impact, and (ii) a menthol-containing solids composition according to the invention (e.g. 0.7 wt. %, based on a finished chewing gum), which has the effect of delayed release of the flavor and therefore the potent “long-lasting” effect, is thus an ideal complementary combination which covers the entire taste spectrum, in particular in chewing gum strips/dragées.

If (a) a peppermint flavor, e.g. a commercially obtainable Optamint® flavor (Symrise GmbH & Co. KG) and (b) a menthol-containing solids composition according to the invention are incorporated in combination into chewing gum strips/dragées, a more pronounced “long-lasting” effect is also to be perceived here.

In the case of dragées, in particular in chewing gum dragées, the taste can be improved decisively if the coating (i.e. the covering, which is multilayered in some cases, applied by dragée-coating) is prepared using a menthol-containing solids composition according to the invention. Corresponding dragées according to the invention cause a very clear, radiant and novel fresh menthol taste during chewing in the mouth. This sensory effect is also achieved by dragée-coating other confectionery, such as e.g. chewing sweets or hard caramels.

A coating preferably comprises at least one sugar (preferably chosen from xylose, fructose, galactose, glucose, mannose, lactose, maltose, sucrose and mixtures thereof; glucose and sucrose are particularly preferred) and/or at least one sugar substitute (preferably chosen from isomaltol, sorbitol, xylitol, maltitol and/or mannitol).

In a further aspect, the present invention therefore relates to a sugar-coated product which is suitable for consumption, characterized in that the coating comprises (a) solid menthol bodies and (b) a solid anticaking agent comprising or (preferably) consisting of particles having a particle size of not more than 100 μm,

wherein the solid anticaking agent is chosen from the group consisting of

calcium phosphate, magnesium phosphate,

magnesium hydroxycarbonate,

magnesium oxide,

mixtures of magnesium oxide and silicon dioxide,

mannitol,

calcium silicate, magnesium silicate, talc,

polydimethylsiloxane,

Al, Ca, K, Na, Mg or NH₄⁺ salts of edible fatty acids

and mixtures thereof,

wherein in at least one layer of the coating the content of the solid anticaking agent (b) is not more than 8 wt. %, based on the total weight of components (a) and (b).

The above statements apply accordingly in respect of preferred solid flow auxiliaries.

The use of the menthol-containing solids compositions according to the invention in dragées and compressed tablets is very advantageous, since at a dosage of only 0.2 wt. % the taste profile is already much clearer and fresher than in the case of the solid forms of menthol which have been used hitherto. A radiant menthol taste is formed, which increases outstandingly to give a “power mint” product when the dosage is increased to 0.5 wt. %. In this context, the percentage data relate to the total weight of the ready-to-use dragées or compressed tablets. In dragées and compressed tablets, the menthol-containing solids compositions according to the invention have the effect of more freshness or an intensification of the fresh taste in the same dosage, compared with other presentation forms.

The menthol-containing solids compositions according to the invention render possible the preparation of glass-type flavor particles by means of extrusion of an aromatized matrix of sugar(s) and/or sugar substitute(s) with a loading of menthol which was hitherto not possible. The preparation and use of glass-type flavor particles is described, for example, in WO 03/092412 and the literature cited there. Hitherto, solid menthol was dissolved in a solvent and incorporated into the extrusion process as a solution. In this context, the maximum menthol concentration which can be achieved in the finished glass-type flavor particles is determined by two limits: on the one hand by the solubility limit of the menthol in the solvent or solvents used, on the other hand by the amount of solvent or solvents used itself or themselves, since because of their softening properties, solvents reduce the extrudability of the aromatized matrix of sugars and/or sugar substitutes, or at too high a dosage prevent extrusion.

According to the invention, the preparation of a solid (homogeneous) premix of sugar(s) and/or sugar substitute(s) and menthol-containing solids composition according to the invention which can be extruded directly, as a result of which menthol loadings of up to 20 wt. % can be achieved, based on the finished glass-type flavor particles, is now possible. The corresponds to a menthol loading 5 to 6 times higher than was hitherto possible in the case of glass-type flavor particles.

In a further aspect, the present invention therefore relates to glass-type flavor particles comprising at least 3.5 wt. %, preferably at least 5 wt. % of menthol, based on the finished glass-type flavor particles, and a solid anticaking agent,

wherein the solid anticaking agent is chosen from the group consisting of

calcium phosphate, magnesium phosphate,

magnesium hydroxycarbonate,

magnesium oxide,

mixtures of magnesium oxide and silicon dioxide,

mannitol,

calcium silicate, magnesium silicate, talc,

polydimethylsiloxane,

Al, Ca, K, Na, Mg or NH₄⁺ salts of edible fatty acids,

and mixtures thereof.

In this context, the above statements apply accordingly in respect of preferred solid flow auxiliaries.

In preferred embodiments, the content of solid anticaking agent in a glass-type flavor particle according to the invention is at least 0.05 wt. %, preferably at least 0.1 wt. %.

The menthol-containing solids composition according to the invention can of course be used not only for the purposes listed above by way of example. The present invention accordingly also quite generally relates to articles which comprise a menthol-containing solids composition according to the invention. In this context, however, the articles are preferably chosen from the group consisting of: products which are suitable for consumption, smoking goods, tobacco products, perfumes (fragrances), flavors, oral hygiene products, cosmetic, pharmaceutical and dermatological products and encapsulated menthol-containing solids compositions.

A related aspect of the present invention accordingly relates to the use of a menthol-containing solids composition according to the invention for the preparation of a menthol-containing article, in particular an article which is chosen from the group consisting of: products which are suitable for consumption, smoking goods, tobacco products, perfumes (fragrances), flavors, oral hygiene products, cosmetic, pharmaceutical and dermatological products and encapsulated menthol-containing solids compositions.

In this context, the menthol-containing solids compositions according to the invention are of course employed in particular for aromatizing or fragrancing the articles mentioned.

The content of the menthol-containing solids composition according to the invention in a product varies considerably according to the product type:

In ready-to-use products, such as, for example, oral hygiene products, products which are suitable for consumption (e.g. foodstuffs) or cosmetic products, the content of the menthol-containing solids composition according to the invention is preferably in the range of 0.01-10 wt. %, particularly preferably in the range of from 0.1 to 5 wt. %, based on the total weight of the ready-to-use product.

In flavor or fragrance mixtures, on the other hand, the content of the menthol-containing solids composition according to the invention can be very much higher, and this is regularly in the range of from 0.01 to 70 wt. %, preferably in the range of from 1 to 50 wt. %, based on the total weight of the flavor or of the fragrance mixture.

A product which is suitable for consumption is a product which is intended to be introduced into the human oral cavity, to remain there for a certain period of time and then either to be swallowed, i.e. eaten (e.g. foodstuffs) or to be removed from the oral cavity again (e.g. chewing gums). This includes all substances or products which are intended to be taken in by humans in the processed, partly processed or non-processed state. This furthermore includes all substances which are added to the product which is suitable for consumption during its preparation, processing or working.

Preferred products which are suitable for consumption are, for example, baked goods (biscuits, cakes, muffins, waffles, baking mixtures), confectionery (hard caramels, soft caramels, chewing sweets, compressed tablets, dragées, sugar beads, sugar fillings), dairy products (yoghurts, puddings, ice-cream), chocolate goods (white, milk or dark chocolate, chocolate bars), creamy compositions (fillings for baked goods, such as e.g. biscuit fillings, creamy fillings for chocolates, creamy fillings for bars), chewing gums sugar-free, sugar-containing, strips, compressed tablets, dragées), snacks and snack mixtures, water-soluble powder products and mixtures for sprinkling on (toppings).

In the context of the invention, an oral hygiene product (also called oral care product or oral hygiene formulation in the following) is understood as meaning one of the formulations familiar to the person skilled in the art for cleansing and care of the oral cavity and the pharyngeal cavity and for refreshing the breath. This expressly includes care of the teeth and gums. Presentation forms of customary oral hygiene formulations are creams, gels, pastes, foams, emulsions, suspensions, aerosols and sprays, and also capsules, granules, pastilles, tablets, sweets or chewing gums, without this list being intended to be understood as limiting for the purpose of this invention.

Preferred oral hygiene are, in particular, dental care compositions, such as toothpastes, dental creams, dental gels, dental powders, mouthwashes, dental floss, seamless capsules, sweets for sucking and sugar-free chewing gums.

A menthol-containing solids composition according to the invention can moreover be further processed by encapsulation. Preferably, the menthol-containing solids composition and/or a liquid or solid formulation comprising this is encapsulated with a solid encapsulating material, which is preferably chosen from starches, degraded or chemically or physically modified starches (in particular dextrins and maltodextrins), gelatines, gum arabic, agar-agar, ghatti gum, gellan gum, modified and non-modified celluloses, pullulan, curdlan, carrageenan, alginic acid, alginates, pectin, inulin, xanthan gum and mixtures of two or more of the substances mentioned.

Preferably, the solid shell material is chosen from gelatine (pig, bovine, chicken and/or fish gelatines and mixtures thereof, preferably comprising at least one gelatine having a Bloom value of greater than or equal to 200, preferably having a Bloom value of greater than or equal to 240, are advantageous), maltodextrin (preferably obtained from maize, wheat, tapioca or potato; preferred maltodextrins have a DE value in the range of 10-20), modified cellulose (e.g. cellulose ethers), alginates (e.g. Na alginate), carrageenan (beta-, iota-, lambda- and/or kappa-carrageenan), gum arabic, curdlan and/or agar-agar. Gelatine is preferably used in particular because of its good availability in various Bloom values.

Seamless gelatine or alginate capsules, the shell of which dissolves very rapidly in the mouth or bursts when chewed and releases the active compound in the oral cavity in this way, are particularly preferred especially for oral hygiene purposes. The preparation can be carried out, for example, as described in EP 0 389 700, JP 7 196 478, U.S. Pat. No. 4,251,195, U.S. Pat. No. 6,214,376, WO 03/055587 or WO 2004/050069.

The cosmetic and/or dermatological products according to the invention can have a conventional composition and can serve for cosmetic and/or dermatological sun protection, and furthermore for the treatment, care and cleansing of the skin and/or hair and as a make-up product in decorative cosmetics. The products according to the invention can correspondingly be used, for example, depending on their build-up, as skin protection cream, cleansing milk, sunscreen lotion, nutrient cream, day or night cream etc. It is possible and advantageous, where appropriate, to use the products according to the invention as a base for pharmaceutical products. Those cosmetic and dermatological products which are in the form of a skin care or make-up product are preferred in particular. Creams, gels, lotions, alcoholic and aqueous/alcoholic solutions, emulsions or stick preparations are a typical embodiment. These compositions can furthermore comprise as further auxiliary substances and additives mild surfactants, co-emulsifiers, superfatting agents, pearlescent waxes, agents for imparting consistency, thickeners, polymers, silicone compounds, fats, waxes, stabilizers, biogenic active compounds, deodorizing active compounds, antidandruff agents, film-forming agents, swelling agents, hydrotopes, preservatives, insect repellents, tanning agents, artificial self-tanning agents (e.g. dihydroxyacetone), solubilizers, perfume oils, dyestuffs, germ-inhibiting agents and the like.

For use, the cosmetic and dermatological products according to the invention are applied to the skin and/or hair in a sufficient amount in the conventional manner for cosmetics.

Those cosmetic and/or dermatological products according to the invention which are in the form of a cosmetic composition for protection of the skin and hair are particularly preferred. These can advantageously comprise at least one inorganic pigment, preferably an inorganic micropigment, in addition to UV-A, UV-B and/or broad band filters used according to the invention.

The cosmetic and/or dermatological products according to the invention can comprise cosmetic auxiliary substances such as are conventionally used in such products, e.g. preservatives, bactericides, perfumes, substances for preventing foaming, dyestuffs, pigments which have a colouring action, thickeners, moisturizing and/or moisture-retaining substances, fats, oils, waxes or other conventional constituents of a cosmetic or dermatological product, such as alcohols, polyols, polymers, foam stabilizers, electrolytes, organic solvents or silicone derivatives.

Further preferred embodiments of the present invention emerge from the attached patent claims and the following examples. In this context, the above statements always apply in respect of preferred solid flow auxiliaries. Unless stated otherwise, the data in the examples relate to the weight.

### EXAMPLE 1

Synthetic I-menthol (from thymol) in the form of freshly prepared needle-shaped crystals (the content of menthol having a melting point of 42-43° C. was >99 wt. %) having the following crystal size distribution (sieve analysis) was employed in the following Examples 1a and 1b:

13 wt. % having a crystal size of less than 0.20 mm;

58 wt. % having a crystal size in the range of from 0.20 mm to 0.50 mm;

19 wt. % having a crystal size in the range of from 0.51 mm to 0.80 mm;

5 wt. % having a crystal size in the range of from 0.81 mm to 1.0 mm;

2 wt. % having a crystal size in the range of from 1.1 mm to 1.25 mm; and

3 wt. % having a crystal size in the range of greater than 1.25 mm.

### EXAMPLE 1a

I-Menthol (25 kg) in the form of freshly prepared needle-shaped crystals (for the particle distribution, see Example 1) was initially introduced into a mixer and mixed briefly and intensively with 380 g magnesium oxide (calculated average particle size: 12.8 nm, specific BET surface area: 130 m²/g). The content of magnesium oxide in the total solids mixture was approx. 1.5%. The original menthol crystals were comminuted by the mixing operation.

The menthol-containing solids composition according to the invention obtained in this way was subjected to a sieve analysis and had the following particle distribution:

49.6 wt. % having a crystal size of less than 0.20 mm;

45 wt. % having a crystal size in the range of from 0.20 mm to 0.50 mm;

3.2 wt. % having a crystal size in the range of from 0.51 mm to 0.80 mm;

2 wt. % having a crystal size in the range of from 0.81 mm to 1.0 mm;

0.1 wt. % having a crystal size in the range of from 1.1 mm to 1.25 mm; and

0.1 wt. % having a crystal size in the range of greater than 1.25 mm.

This menthol-containing solids composition was packed in a plastic bag in a cube-shaped box and stored at 20° C. (bulk height: 50 cm). Every 14 days, the composition according to the invention was investigated optically and mechanically for its flowability. No caking or formation of lumps was to be found even after nine months.

### EXAMPLE 1b

150-200 kg I-menthol in the form of freshly prepared needle-shaped crystals (for the particle distribution, see Example 1) were fed in the course of 1 hour over a vibrating channel to a compactor apparatus (from Hosokawa Bepex GmbH) comprising two compactor rolls having profiled surfaces (circumference 60 cm, width 10 cm, area 600 cm²). At room temperature with a pressing force of 50 kN, the compactor produced pressed pieces in the form of pellets (pillow shape) having the dimensions of length=width=10.2 mm and height=5 mm.

50 kg of these pillow-shaped pellets were mixed with 350 g magnesium oxide (calculated average particle size: 36 nm, specific BET surface area: 46 m²/g).

The menthol-containing formulation prepared in this way showed no formation of lumps or caking even after a storage time of 12 months at a bulk height of 100 cm at room temperature.

### EXAMPLE 1c

I-Menthol (25 kg) in the form of freshly prepared needle-shaped crystals (for the particle distribution, see Example 1) was initially introduced into a mixer and mixed briefly and intensively with various amounts of magnesium oxide (calculated average particle size: 12.8 nm, specific BET surface area: 130 m²/g, compare Example 1a). The content of magnesium oxide (solid anticaking agent) in the total solids mixture was 0.5 wt. %, 1 wt. %, 2 wt. % or 4 wt. %. The original menthol crystals were comminuted by the mixing operation (compare Example 1a). The menthol-containing solids compositions having various contents of magnesium oxide were each packed in a plastic bag in a cube-shaped box and stored at 20° C. (bulk height: 50 cm). After two months, caking or formation of lumps was to be found in none of the total of four compositions. The particular solids compositions still showed a very good flowability even after two months.

### EXAMPLE 1d

Table 1 shows the results of a flowability investigation after storage for three months at 20° C. with menthol crystals (bulk height: 50 cm) having a particle size distribution according to Example 1a. The percentage contents of the particular solid anticaking agent consisting of magnesium oxide and silicon dioxide relate to the menthol-containing composition consisting of menthol crystals and solid anticaking agent. The following qualities (types) of magnesium oxide were employed here as nanopowders:

MgO^{(a)}:

average particle size: 12.8 nm, specific BET surface area: 130 m²/g), source: Sigma-Aldrich

MgO^{(b)}:

average particle size: 20 nm, specific BET surface area: >50 m²/g), source: Accumet Materials Inc.

MgO^{(c)}:

average particle size: 36 nm, specific BET surface area: 46 m²/g), source: Accumet Materials Inc.

TABLE 1

| | | | 1 wt. | 2 wt. | |
|---|---|---|---|---|---|
| | | | % of | % of | 4 wt. % |
| | MgO | SiO₂:MgO | flow | flow | of flow |
| SiO₂ type | type | (w/w) | agent | agent | agent |
| Aerosil ® 805 (150 m²/g; | MgO^{(a)} | 1:3 | A | A | A |
| 12 nm), hydrophobic | | | | | |
| Aerosil ® 812 (260 m²/g; | MgO^{(c)} | 1:1 | A | A | A |
| 7 nm), hydrophobized | | | | | |
| with trimethylsilyl groups | | | | | |
| Aerosil ® 972 (110 m²/g; | MgO^{(b)} | 1:2 | A | A | A |
| 16 nm), hydrophobized | | | | | |
| with dimethylsilyl groups | | | | | |
| Aerosil ® 150 (150 m²/g; | MgO^{(a)} | 1:2 | A | A | A |
| 14 nm), hydrophilic | | | | | |
| Aerosil ® 200 (200 m²/g; | MgO^{(a)} | 2:1 | A | A | A |
| 12 nm), hydrophilic | | | | | |
| Aerosil ® 300 (300 m²/g; | MgO^{(b)} | 1:1 | A | A | A |
| 7 nm), hydrophilic | | | | | |

The data in parentheses relate to the specific surface area in m²/g measured by the BET method (in accordance with DIN 66131) and the average size of the primary particles.
D = severe lump formation, no flowability
C = moderate lump formation, poor flowability
B = free flowability, scarcely any lump formation
A = very good flowability, no lump formation

### EXAMPLE 2

The various rates of solution of a solids composition according to the invention according to Example 1a above and forms of I-menthol which are not according to the invention were compared.

For this, in each case 5 g I-menthol or 5 g of the I-menthol-containing solids composition according to the invention according to Example 1a were added to 95 g of a mixture, temperature-controlled at a constant 20° C., of equal weight contents of ethanol and water and the time taken for complete dissolving (optical determination) of the menthol and of the menthol-containing solids composition was determined.

The particular solution test was carried out in a glass beaker having a diameter of 5 cm and a filling height of 7 cm. The contents of the glass beaker were in each case stirred with a 4 cm long magnetic stirring fish at a magnetic stirrer speed of 4 revolutions per second during the solution test. The temperature was monitored with a Pt-100 thermometer, which was immersed 5 cm deep into the liquid, was placed at a distance of 1 cm from the glass beaker wall and simultaneously served as a baffle.

The crystals of natural menthol which were employed in the solution test had a content of I-menthol of 99.2% and a menthol-free peppermint oil content of 0.8% (data in GC percentage area). The size of the crystals here corresponded to the typical (commercially available) crystal geometry of natural I-menthol.

The following Table 2 shows the dissolving times for the various forms of I-menthol which are not according to the invention in comparison with that of the menthol-containing solids composition from Example 1a.

TABLE 2

| | Time taken for |
|---|---|
| | complete dissolving |
| Menthol pellets according to WO 03/101924, | 504 sec |
| Example 1 | |
| Crystals of natural menthol | 255 sec |
| (rod-shaped crystals, length 15 to 20 mm, | |
| diameter 2 to 4 mm) | |
| Menthol crystals from Example 1 | 170 sec |
| Menthol crystals in lump form, obtained by | 280 sec |
| storage of the menthol crystals from Example | |
| 1 for 2 months | |
| Menthol-containing composition according to | 61 sec |
| the invention from Example 1a (1.5 wt. % | |
| MgO) | |

FORMULATION EXAMPLES F1-F14

F1. Gel dental cream

| | I (%) | II (%) | III (%) |
|---|---|---|---|
| Na carboxymethylcellulose | 0.40 | 0.40 | 0.40 |
| Sorbitol 70%, in water | 72.00 | 72.00 | 72.00 |
| Polyethylene glycol (PEG) 1500 | 3.00 | 3.00 | 3.00 |
| Na saccharinate | 0.07 | 0.07 | 0.07 |
| Na fluoride | 0.24 | 0.24 | 0.24 |
| p-Hydroxybenzoic acid (PHB) ethyl | 0.15 | 0.15 | 0.15 |
| ester | | | |
| Peppermint oil flavor | 0.90 | 0.56 | 0.30 |
| Menthol-containing composition from | 0.125 | 0.50 | 0.90 |
| Example 1a | | | |
| Abrasive silica | 11.00 | 11.00 | 11.00 |
| Thickening silica | 6.00 | 6.00 | 6.00 |
| Sodium dodecyl sulfate (SDS) | 1.40 | 1.40 | 1.40 |
| Dist. water | to 100.00 | to 100.00 | to 100.00 |

F2. Dental cream against plaque

| | I (%) | II (%) | III (%) |
|---|---|---|---|
| Na carboxymethylcellulose | 1.00 | 1.00 | 1.00 |
| Glycerol | 12.50 | 12.50 | 12.50 |
| Sorbitol 70%, in water | 29.00 | 29.00 | 29.00 |
| Na saccharinate | 0.20 | 0.20 | 0.20 |
| Na fluoride | 0.22 | 0.22 | 0.22 |
| Azacycloheptane-2,2-diphospho acid, | 1.00 | 1.00 | 1.00 |
| di-sodium salt | | | |
| Bromochlorophene | 0.10 | 0.10 | 0.10 |
| Spearmint flavor | 1.00 | 0.60 | 0.20 |
| Menthol-containing composition from | 0.125 | 0.56 | 1.00 |
| Example 1a | | | |
| Abrasive silica | 15.00 | 15.00 | 15.00 |
| Thickening silica | 5.00 | 5.00 | 5.00 |
| Sodium dodecyl sulfate (SDS) | 1.50 | 1.50 | 1.50 |
| Dist. water | to 100.00 | to 100.00 | to 100.00 |

F3. Dental cream against plaque

| | I (%) | II (%) | III (%) |
|---|---|---|---|
| Carrageenan | 0.90 | 0.90 | 0.90 |
| Glycerol | 15.00 | 15.00 | 15.00 |
| Sorbitol 70%, in water | 25.00 | 25.00 | 25.00 |
| PEG 1000 | 3.00 | 3.00 | 3.00 |
| Na fluoride | 0.24 | 0.24 | 0.24 |
| Tetrapotassium diphosphate | 4.50 | 4.50 | 4.50 |
| Tetrasodium diphosphate | 1.50 | 1.50 | 1.50 |
| Na saccharinate | 0.40 | 0.40 | 0.40 |
| Precipitated silica | 20.00 | 20.00 | 20.00 |
| Titanium dioxide | 1.00 | 1.00 | 1.00 |
| PHB methyl ester | 0.10 | 0.10 | 0.10 |
| Eucalyptus/spearmint flavor | 1.00 | 0.60 | 0.20 |
| Menthol-containing | 0.125 | 0.56 | 1.00 |
| composition from Example | | | |
| 1a | | | |
| Sodium dodecyl sulfate | 1.30 | 1.30 | 1.30 |
| Dist. water | to 100.00 | to 100.00 | to 100.00 |

F4. Dental cream for care of sensitive teeth

| | I (%) | II (%) | III (%) |
|---|---|---|---|
| Na carboxymethylcellulose | 0.70 | 0.70 | 0.70 |
| Xanthan gum | 0.50 | 0.50 | 0.50 |
| Glycerol | 15.00 | 15.00 | 15.00 |
| Sorbitol 70%, in water | 12.00 | 12.00 | 12.00 |
| K nitrate | 5.00 | 5.00 | 5.00 |
| Na monofluorophosphate | 0.80 | 0.80 | 0.80 |
| PHB methyl ester | 0.15 | 0.15 | 0.15 |
| PHB propyl ester | 0.05 | 0.05 | 0.05 |
| Na saccharinate | 0.20 | 0.20 | 0.20 |
| Herb flavor | 1.00 | 0.60 | 0.20 |
| Menthol-containing composition | 0.125 | 0.56 | 1.00 |
| from Example 1a | | | |
| Ca carbonate | 35.00 | 35.00 | 35.00 |
| Silicon dioxide | 1.00 | 1.00 | 1.00 |
| Sodium dodecyl sulfate (SDS) | 1.50 | 1.50 | 1.50 |
| Dist. water | to 100.00 | to 100.00 | to 100.00 |

F5. Dental cream for care of sensitive teeth

| | I (%) | II (%) | III (%) |
|---|---|---|---|
| Hydroxyethylcellulose | 1.40 | 1.40 | 1.40 |
| Guar gum | 0.60 | 0.60 | 0.60 |
| Glycerol | 18.00 | 18.00 | 18.00 |
| Sorbitol 70%, in water | 12.00 | 12.00 | 12.00 |
| Na saccharinate | 0.35 | 0.35 | 0.35 |
| Dyestuff | 0.01 | 0.01 | 0.01 |
| PHB methyl ester | 0.15 | 0.15 | 0.15 |
| PHB propyl ester | 0.04 | 0.04 | 0.04 |
| Sr chloride | 10.50 | 10.50 | 10.50 |
| Cinnamon flavor | 1.00 | 0.60 | 0.30 |
| Menthol-containing | 0.225 | 0.56 | 1.00 |
| composition from Example 1a | | | |
| Precipitated silica | 15.00 | 15.00 | 15.00 |
| Silicon dioxide | 1.60 | 1.60 | 1.60 |
| Sodium dodecyl sulfate | 1.30 | 1.30 | 1.30 |
| Dist. water | to 100.00 | to 100.00 | to 100.00 |

F6. Ready-to-use mouth wash with fluoride

| | I (%) | II (%) | III (%) |
|---|---|---|---|
| Ethanol | 7.00 | 7.00 | 7.00 |
| Glycerol | 12.00 | 12.00 | 12.00 |
| Na fluoride | 0.05 | 0.05 | 0.05 |
| Pluronic F-127 ® (BASF, | 1.40 | 1.40 | 1.40 |
| surface-active substance) | | | |
| Na phosphate buffer pH 7.0 | 1.10 | 1.10 | 1.10 |
| Sorbic acid | 0.20 | 0.20 | 0.20 |
| Na saccharinate | 0.10 | 0.10 | 0.10 |
| Thymol/wintergreen flavor | 1.00 | 0.60 | 0.20 |
| Menthol-containing | 0.125 | 0.56 | 1.00 |
| composition from Example 1a | | | |
| Dyestuff | 0.01 | 0.01 | 0.01 |
| Dist. water | to 100.00 | to 100.00 | to 100.00 |

F7. Mouth wash concentrate with an activity against bad breath

| | I (%) | II (%) | III (%) |
|---|---|---|---|
| Ethanol, 95% strength | 80.00 | 80.00 | 80.00 |
| Na cyclamate | 0.15 | 0.15 | 0.15 |
| Spearmint/thymol flavor | 3.50 | 3.50 | 3.50 |
| Dyestuff | 0.01 | 0.01 | 0.01 |
| Menthol-containing | 0.50 | 1.0 | 3.0 |
| composition from Example 1a | | | |
| Dist. water | to 100.00 | to 100.00 | to 100.00 |

F8. Chewing gum, sugar-containing

| | I (%) | II (%) | III (%) |
|---|---|---|---|
| Chewing gum base | 21.00 | 21.00 | 21.00 |
| Glucose syrup | 16.50 | 16.50 | 16.50 |
| Glycerol | 0.50 | 0.50 | 0.50 |
| Powdered sugar | 60.45 | 60.40 | 60.30 |
| Spearmint flavor | 1.40 | 1.00 | 0.50 |
| Menthol-containing | 0.15 | 0.60 | 1.20 |
| composition from Example 1a | | | |

F9. Sugar-free chewing gum

| | I (%) | II (%) | III (%) |
|---|---|---|---|
| Chewing gum base | 30.00 | 30.00 | 30.00 |
| Sorbitol, powder | 38.45 | 38.40 | 38.30 |
| Palatinite | 9.50 | 9.50 | 9.50 |
| Xylitol | 2.00 | 2.00 | 2.00 |
| Mannitol | 3.00 | 3.00 | 3.00 |
| Aspartame | 0.10 | 0.10 | 0.10 |
| Acesulfame K | 0.10 | 0.10 | 0.10 |
| Emulgum/emulsifier | 0.30 | 0.30 | 0.30 |
| Sorbitol 70%, in water | 14.00 | 14.00 | 14.00 |
| Glycerol | 1.00 | 1.00 | 1.00 |
| Cinnamon/menthol flavor | 1.40 | 1.00 | 0.50 |
| Menthol-containing | 0.15 | 0.60 | 1.20 |
| composition from Example 1a | | | |

F10. Chewing gum dragees, sugar-free
Q1: Chewing gum base composition constituent

| | I (%) | II (%) | III (%) |
|---|---|---|---|
| Chewing gum base | 37.00 | 37.00 | 37.00 |
| Sorbitol, powder | 50.50 | 50.50 | 50.50 |
| Aspartame | 0.20 | 0.20 | 0.20 |
| Plasticizer (Emulgum) | 0.50 | 0.50 | 0.50 |
| Acesulfame K | 0.20 | 0.20 | 0.20 |
| Sorbitol 70% in water | 5.00 | 5.00 | 5.00 |
| Glycerol | 4.00 | 4.00 | 4.00 |
| Peppermint oil flavor | 1.60 | 1.60 | 1.60 |
| (Optamint ®, Symrise) | | | |
| Menthol spray-dried | 1.00 | 1.00 | 1.00 |

All the constituents of the chewing gum base composition (Q1) were mixed, stamped into chewing gum strands and then shaped into individual chewing gum pillows. The chewing gum pillows were then wetted (gummed) with a 40 wt. % strength gum arabic solution in a rotating dragée-coating drum. The gummed chewing gum pillows were then coated in a rotating dragée-coating drum with the pulverulent mixture A, which substantially comprised the menthol-containing solids composition according to the invention and at least one sugar substitute (usually chosen from isomaltol, sorbitol, xylitol, maltitol and/or mannitol, pulverulent gum arabic can optionally additionally be used). After adequate drying with cold air, the chewing gum pillows coated in this way were dried overnight. For further application of the coating to the dried, coated chewing gum pillows using coating solution B, 15 layers were first applied by means of dragée-coating, and in the 16th layer a mixture of constituent C and mixture B was applied. Thereafter, further layers were applied using mixture B, until the total weight of the coating (Q2) was about 35 wt. % of the weight of the original chewing gum pillows (Q1). In order to impart gloss to the chewing gum dragées, a final treatment was carried out with a polishing agent, which comprised a mixture of equal weight contents of carnauba wax and beeswax. The ready-to-use chewing gum dragées cause a very clear, radiant, fresh and novel menthol taste during chewing in the mouth.

Q2: Coating constituent (covering)

(the weight contents stated relate to the total weight of the coating (Q2) applied to the chewing gum pillows (Q1); the total weight of Q2 was about 35%, based on the weight of Q1)

| | I (%) | II (%) | III (%) |
|---|---|---|---|
| Mixture A | | | |
| Isomaltol | 0.20 | 0 | 0 |
| Sorbitol | 0 | 0.40 | 0 |
| Mannitol | 0 | 0 | 0.80 |
| Menthol-containing composition from | 0.20 | 0.60 | 1.00 |
| Example 1a | | | |
| Mixture B | | | |
| Isomaltol | 68.00 | 67.70 | 67.40 |
| Water | 26.7 | 26.6 | 26.5 |
| Gum arabic 40% in water (this | 2.50 | 2.50 | 2.50 |
| content includes the amount used for | | | |
| the gumming) | | | |
| Acesulfame K | 0.05 | 0.05 | 0.05 |
| Aspartame | 0.05 | 0.05 | 0.05 |
| Titanium dioxide | 1.50 | 1.50 | 1.50 |
| Constituent C | | | |
| Peppermint oil flavor (Optamint ®, | 0.80 | 0.60 | 0.20 |
| Symrise) | | | |

F11. Gelatine capsule for direct consumption

| | I (%) | II (%) | III (%) |
|---|---|---|---|
| Gelatine shell: | | | |
| Glycerol | 2.014 | 2.014 | 2.014 |
| Gelatine 240 Bloom | 7.91 | 7.91 | 7.91 |
| Sucralose | 0.065 | 0.065 | 0.065 |
| Allura Red | 0.006 | 0.006 | 0.006 |
| Brilliant Blue | 0.005 | 0.005 | 0.005 |
| Core composition: | | | |
| Plant oil triglyceride | 85.0 | 80.0 | 73.0 |
| Flavor B | 4.0 | 6.0 | 10.0 |
| Menthol-containing | 1.0 | 4.0 | 7.0 |
| composition from Example 1a | | | |

Flavor B here had the following composition (data in each case in wt. %):

0.1% neotame powder, 0.05% aspartame, 29.3% lemon oil, 29.3% orange oil, 2.97% sucralose, 2.28% triacetin, 5.4% diethyl tartrate, 12.1% peppermint oil yakima, 0.7% ethanol, 3.36% 2-hydroxyethyl menthyl carbonate, 3.0% 2-hydroxypropyl menthyl carbonate, 0.27% vanillin, 5.5% d-limonene, 5.67% I-menthyl acetate.

The gelatine capsule, which is suitable for direct consumption, had a diameter of 5 mm, and the weight ratio of core material to shell material was 90:10. The capsules opened in the mouth within less than 10 seconds and dissolved completely within less than 50 seconds.

F12. Chewing sweet

| | | |
|---|---|---|
| Water | | 7.5% |
| Sugar | refined sugar C4 | 41.2% |
| Glucose syrup | dextrose 40 | 36.2% |
| Hydrogenated plant fat | melting point 32-36° C. | 6.5% |
| Lecithin | emulsifier (soya lecithin) | 0.3% |
| Gelatine | pig gelatine | 0.8% |
| Fondant | type S30 | 4.8% |
| Lemon flavor | | 0.6% |
| Menthol-containing composition from | | 2.1% |
| Example 1a | | |

Preparation instructions:
a) swell the gelatine with water (1.8 times the amount of gelatine) at 70° C. for 2 hours;
b) boil the sugar, syrup, water, fat and lecithin at 123° C.;
c) slowly mix the gelatine solution with the boiling mixture;
d) stir in the menthol-containing composition and optionally colour;
e) control the resulting mass at a temperature of approx. 70° C. on a cooling table, then add the fondant and aerate on a drawing machine for approx. 3 minutes;
f) subsequently cut the chewing sweet mass and package.

When the chewing sweet is eaten, a strong menthol taste is perceived during the chewing and the texture of the chewing sweet is pleasant.

F13. Compressed tablet, sugar-containing or sugar-free

| | | |
|---|---|---|
| Dextrose (sugar-containing) or sorbitol | | 98.5-98.8% |
| (sugar-free) | | |
| Magnesium stearate | lubricant | 1.0% |
| Menthol-containing composition from | | 0.2-0.5% |
| Example 1a | | |

Mix all the constituents and press to compressed tablets in a suitable machine.

F14. Extrudate

| | | |
|---|---|---|
| Glucose syrup, spray-dried | Glucidex IT33W | 60.0% |
| (DE value: 31-34) | (Roquette) | |
| Maltodextrin (DE value: 17-20) | (Cerestar) | 26.0% |
| Emulsifier Monomuls | emulsifier based on | 1.5% |
| | hydrogenated palm oil; | |
| | melting point: 64° C., | |
| | (Grünau) | |
| Dextrose monohydrate (DE value: 99.5) | dextrose, containing | 1.5% |
| | water of crystallization | |
| | (Cerestar) | |
| Water | | 1.5% |
| Menthol-containing composition from | | 9.5% |
| Example 1a | | |

Preparation instructions (see also WO 03/092412):

All the constituents were mixed and the mixture was conveyed in a twin-screw extruder by one-point metering. The extrusion temperatures were between 100 and 120° C. and the specific energy input was 0.2 kWh/kg. The strands emerging from the extruder die plate, which was provided with 1 mm bores, were cut to particles of approx. 1 mm diameter by rotating blades immediately after exit from the dies. The granules prepared in this way had a menthol content of 10 wt. %.

1. Menthol-containing solids composition comprising:
(a) a solid menthol component including bodies having a menthol content of 95 wt. % or more, based on the total weight of the bodies, and
(b) a solid anticaking agent, including particles having a particle size of not more than 100 μm,
wherein particles of the anticaking agent adhere to a surface of the bodies of the solid menthol component,
wherein the solid anticaking agent is
calcium phosphate, magnesium phosphate,
magnesium hydroxycarbonate,
magnesium oxide,
mixtures of magnesium oxide and silicon dioxide,
mannitol,
calcium silicate, magnesium silicate, talc,
polydimethylsiloxane,
Al, Ca, K, Na, Mg or NH₄⁺ salts of edible fatty acids,
or mixtures thereof,
and wherein the content of the solid anticaking agent is not more than 8 wt. % and the content of the solid menthol component is at least 91 wt. %, in each case based on the total weight of the solids composition.

2. Menthol-containing solids composition according to claim 1,
wherein the solid anticaking agent is
calcium phosphate, magnesium phosphate,
magnesium hydroxycarbonate,
magnesium oxide,
mixtures of magnesium oxide and silicon dioxide,
calcium silicate, magnesium silicate, talc,
polydimethylsiloxane,
Ca, K or Na salts of edible fatty acids,
or mixtures thereof.

3. Menthol-containing solids composition according to claim 1, wherein the bodies of the solid menthol component include between 0 and 1% of inorganic constituents, based on the total weight of the bodies.

4. Menthol-containing solids composition according to claim 1, wherein the bodies of the solid menthol component are in the form of crystals, prills, flakes, pressed pieces, pastilles, granules, powders and/or dust.

5. Menthol-containing solids composition according to claim 1, wherein the bodies of the menthol component do not contain surfactants, do not contain
peppermint oil on their surface and
include not more than 0.2 wt. % of water, based on the total weight of the solids composition.

6. Menthol-containing solids composition according to claim 1, wherein particles of the solid anticaking agent adhere to and cover the surface of the bodies of the solid menthol component such that the sublimation capacity or the vapour pressure of the solid menthol from the bodies of the menthol component at 25° C. under 1,013 mbar is reduced by at least 25% compared with the pure menthol component (a).

7. Menthol-containing solids composition according to claim 1, wherein the content of the solid anticaking agent in the solids composition is not more than 4 wt. %, based on the total weight of the solids composition.

8. (canceled)

9. Menthol-containing solids composition according to claim 1, further comprising a product which is suitable for consumption, smoking, a tobacco, a perfume or fragrance an oral hygiene product, a cosmetic, a pharmaceutical or dermatological product or an encapsulated material.

10. Sugar-coated product which is suitable for consumption, having a coating comprising:
(a) solid menthol bodies; and
(b) a solid anticaking agent, including particles having a particle size of not more than 100 μm,
wherein the solid anticaking agent is
calcium phosphate, magnesium phosphate,
magnesium hydroxycarbonate,
magnesium oxide,
mixtures of magnesium oxide and silicon dioxide,
mannitol,
calcium silicate, magnesium silicate, talc,
polydimethylsiloxane,
Al, Ca, K, Na, Mg or NH₄⁺ salts of edible fatty acids
or mixtures thereof,
wherein in at least one layer of the coating the content of a solid anticaking agent is not more than 8 wt. %, based on the total weight of components (a) and (b).

11. Glass-type flavor particles comprising at least 3.5 wt. %, preferably at least 5 wt. % of menthol, based on the finished glass-type flavor particles, and a solid anticaking agent, wherein the solid anticaking agent is
calcium phosphate, magnesium phosphate,
magnesium hydroxycarbonate,
magnesium oxide,
mixtures of magnesium oxide and silicon dioxide,
mannitol,
calcium silicate, magnesium silicate, talc,
polydimethylsiloxane,
Al, Ca, K, Na, Mg or NH₄⁺ salts of edible fatty acids,
or mixtures thereof.

12. A menthol-containing solids composition product which is suitable for consumption, smoking, a tobacco product, a perfume or fragrance, a flavouring agent, an oral hygiene product, a cosmetic, a pharmaceutical or dermatological product or an encapsulated menthol-containing solids compositions comprising:
(a) a solid menthol component including bodies having a menthol content of 95 wt. % or more, based on the total weight of the bodies, and
(b) a solid anticaking agent, including particles having a particle size of not more than 100 μm,
wherein particles of the anticaking agent adhere to a surface of the bodies of the solid menthol component,
wherein the solid anticaking agent is
calcium phosphate, magnesium phosphate,
magnesium hydroxycarbonate,
magnesium oxide,
mixtures of magnesium oxide and silicon dioxide,
mannitol,
calcium silicate, magnesium silicate, talc,
polydimethylsiloxane,
Al, Ca, K, Na, Mg or NH₄⁺ salts of edible fatty acids,
or mixtures thereof,
and wherein the content of the solid anticaking agent is not more than 8 wt. % and the content of the solid menthol component is at least 91 wt. %, in each case based on the total weight of the solids composition.

13. A method for preparing menthol-containing solids composition comprising; (a) a solid menthol component including bodies having a menthol content of 95 wt. % or more, based on the total weight of the bodies, and
(b) a solid anticaking agent, including particles having a particle size of not more than 100 μm,
wherein particles of the anticaking agent adhere to a surface of the bodies of the solid menthol component,
wherein the solid anticaking agent is
calcium phosphate, magnesium phosphate,
magnesium hydroxycarbonate,
magnesium oxide,
mixtures of magnesium oxide and silicon dioxide,
mannitol,
calcium silicate, magnesium silicate, talc,
polydimethylsiloxane,
Al, Ca, K, Na, Mg or NH₄⁺ salts of edible fatty acids,
or mixtures thereof,
comprising the step of
mixing
(a) said solid menthol containing solids composition exhibiting a menthol content of 95 wt. % or more, based on the total weight of the bodies, and
(b) said solid anticaking agent, exhibiting particles having a particle size of not more than 100 μm, until particles of the solid anticaking agent adhere to the surface of the bodies of the menthol component,
wherein the content of the solid anticaking agent is not more than 4 wt. % and the content of the menthol component is at least 95 wt. %, in each case based on the total weight of the solids composition prepared.

14. A method of reducing or preventing sublimation of menthol and caking of bodies of a solid menthol component having a menthol content of 95 wt. % or more based on the total weight of the bodies, by adding a solid anticaking agent that is
calcium phosphate, magnesium phosphate,
magnesium hydroxycarbonate,
magnesium oxide,
mixtures of magnesium oxide and silicon dioxide,
mannitol,
calcium silicate, magnesium silicate, talc,
polydimethylsiloxane,
Al, Ca, K, Na, Mg or NH₄⁺ salts of edible fatty acids
or mixtures thereof, to the solid menthol component.
